(19) United States
(12) Patent Application Publication
(10) Pub. No.: US 2025/0114158 A1
(43) Pub. Date: Apr. 10, 2025

(54) ROBOTIC COMPILATION OF MULTIPLE NAVIGATION MARKERS

(71) Applicant: LEM Surgical AG, Bem (CH)

(72) Inventor: Yossi BAR, Muri bei Bern (CH)

(73) Assignee: LEM Surgical AG, Bem (CH)

(21) Appl. No.: 18/986,447

(22) Filed: Dec. 18, 2024

Related U.S. Application Data
(63) Continuation-in-part of application No. PCT/IB2022/058991, filed on Sep. 22, 2022.
(60) Provisional application No. 63/615,076, filed on Dec. 27, 2023.
(60) Provisional application No. 63/355,016, filed on Jun. 23, 2022.

(51) Int. Cl. A61B 34/30; A61B 90/00

(52) U.S. Cl. CPC A61B 34/30; A61B 90/39; A61B 2034/306; A61B 2090/374; A61B 2090/3762; A61B 2090/3983

(57) ABSTRACT

Systems and methods for surgical robotic navigation include multiple small surgical markers which avoid interfere with line of sight and do not otherwise disturb the surgical staff, while providing a convenient and highly accurate methodology for tracking and compiling the markers. Multi-arm robotic surgery systems are described in various embodiments that hold surgical tools and navigation cameras and optimally make use of several small surgical markers placed on patient anatomy of interest, surgical tools and the robotic arms. The small surgical markers are mathematically compiled so that the navigation cameras see a larger “compiled” surgical marker, thus providing greater accuracy.

### CROSS-REFERENCE TO RELATED APPLICATION

This application claims the benefit of U.S. Provisional Application No. 63/615,076 (Attorney Docket No. 67551-709.102), filed on Dec. 27, 2023, and is a continuation-in-part of PCT Application No. PCT/IB2022/058991 (Attorney Docket No. 67551-709.601), filed on Sep. 22, 2022, which claimed the benefit of U.S. Provisional Patent Application 63/355,016 (Attorney Docket No. 67551-709.101), filed on Jun. 23, 2022, the full disclosures of which are incorporated herein by reference.

### BACKGROUND

Field. The present disclosure relates generally to medical devices and methods, and more particularly to systems and methods for surgical robotic navigation using multiple navigation markers to accurately track anatomy and tools in a surgical space.

Robotic navigation technology using a camera or other sensor to track navigation markers, fiducials, or the like, present in the robotic space is well known and implemented in robotic surgical applications, including spinal robotic surgery. For example, a camera or other sensor is located proximate the patient, typically being mounted on a cart, or stand or attached to a wall, ceiling, or the like. One or more navigation markers are located on the relevant patient anatomy, e.g., vertebrae in a robotic spinal procedure. Additional navigation markers can be attached to one or more robot arms or surgical tools that are used in the procedure, and the camera or other sensor of navigation system can track the absolute and/or relative locations of the various marked objects as they move within the surgical space.

The use of navigation markers in robotic surgeries, however, suffers from certain shortcomings. For example, the need for surgeons to have free access to the patient limits the size of navigation markers that can be affixed to the patient anatomy. The need to use smaller navigation markers, in turn, limits that accuracy with which the markers can be tracked by a remote camera or other sensor. While moving the cameras closer to the surgical site could increase the tracking accuracy, the camera or other sensor itself can becomes problematic as it is moved closer to the patient.

Navigation and registration markers used in robotic surgeries are typically usually large, typically having a minimum dimension of at least 7 cm to 10 cm to provide an accuracy of 1 mm to 2 mm at tool tip when the camera is placed at a standard distance from the patient from about 1.5 m to 2.5 meters. Placing several of these large and bulky navigation markers in a surgical space is very limiting and disruptive to the surgeon. Large markers are also prone to distortion and displacement during the course of a robotic surgical procedure, further limiting their accuracy. Large markers are also more susceptible to deflection and dislocation during a procedure, limiting or eliminating any improved accuracy that they might otherwise provide.

While some of these problems might be lessened by using only a single marker for initial registration and subsequent tracking, many surgeries require two, three, four, or more markers.

For example, multiple markers are needed for certain spinal procedures where at least one marker must be placed on each of two, three, or more adjacent vertebra to track relative motion between different vertebrae.

In other cases, multiple tools are required to complete even simple robotic surgeries, and every tool typically requires its own individual marker. The use of multiple large navigation markers to provide the needed accuracy while using relatively distant camera is highly problematic. Not only does the proximity of multiple large markers in a small surgical area interfere with the surgeon's vision and access, and the ability of a single navigation camera to capture all of the large markers in a single frame is also an issue, often referred to as the “line of sight problem.”

For all of these reasons, it would be desirable to provide surgical robotic systems and methods that can provide accurate tracking using a remote camera or other sensor and small markers, particularly multiple small markers. Such systems and methods should be useful in all robotic surgical procedure where multiple large markers might otherwise be deployed and will be particularly useful in robotic spinal surgeries where multiple individual markers are often placed on a plurality of vertebrae. At least some of these objectives are met by the technology described and claimed herein.

Listing of Background Art. Relevant commonly owned publications and application include WO2022/195460; WO2023/067415; WO2023/118984; WO2023//118985; WO2023/144602; WO2023/152561; WO2023/223215; WO2023/237922; PCT/IB2023/055439; PCT/IB2023/056911; PCT/IB2023/055662; PCT/IB2023/055663; U.S. 63/524,911; and 63/532,753, the full disclosures of which are incorporated herein by reference.

### SUMMARY

The systems and methods of the present disclosure may include the implantation of two or more physical surgical navigation markers on a bony or other patient anatomy to allow a controller of a surgical robot to calculate or compile a single virtual marker which appears to be larger than either physical marker when tracked by a camera or other sensor in a surgical robotic space. The use of smaller physical markers is advantageous as such markers are less likely to interfere with a surgeon's line of sight or otherwise disturb performance of the surgical procedure. The systems and methods of the present disclosure further provide for tracking the virtual marker by optically or otherwise sensing the positions of the physical markers using the cameras or other sensors controlled and manipulated by the controller of the surgical robot.

For example, in spinal and other surgical robotic systems, multiple robotic arms may be operating in the surgical field and may each be holding surgical tools, while all in close proximity to surgical staff and the patient. Multiple large surgical markers can be difficult to deploy and track, and current disclosure allows the deployment, tracking and compiling of multiple, small surgical markers into a larger virtual marker which can be used by the surgical robot to accurately track the patient anatomy while minimizing the size of the implanted physical markers.

Accordingly, provided herein are a system and method for tracking and compiling multiple, small markers in a robotic surgical system with navigation capabilities. In some embodiments, a multi-arm surgical robotic system is provided comprising at least two robotic arms. In the multi-arm surgical robotic system, at least one arm is responsible for surgical tasks and at least one arm is used to carry and operate at least one camera as part of a robotic navigation system. The at least two robotic arms are mounted on a single chassis that houses a central controller that governs movement of the robotic arms. In some systems and methods, several small markers may be deployed in the surgical field and may function as individual markers or as an array of markers that can be registered to the anatomical region and tracked by the navigation capabilities of the surgical robotic system. In some embodiments, the markers may be robotically and mathematically compiled such that they form one large marker in the surgical field but nevertheless each individual marker cause minimal interference or obstruction with the surgical operation.

Accordingly, in some embodiments, a system for tracking and compiling multiple, small surgical markers is provided. The system comprises at least two robotic arms based on a single point of origin with a central control unit coordinating the movement of the arms. At least one of the robotic arms carries at least one surgical tool and at least one of the robotic arms carries at least one navigation camera/sensor. Optionally, at least one surgical marker may be placed on at least one arm carrying the surgical tool or on the surgical tool itself. One or more small (e.g. 1-5 cm) surgical markers may be placed in the surgical field on the patient anatomy of interest. In one example, one or more small surgical markers are placed on multiple vertebrae or multiple vertebrae aspects. In various examples, the surgical navigation camera/sensor may be held and manipulated closer than conventional surgical navigation cameras, for example at 1 meter or less from the patient anatomy of interest. This system is able to track the patient anatomy of interest, and optionally a surgical tool, without unduly interfering with the surgical field or the surgeon's view of the field, due to the use of several small markers.

Accordingly, in another embodiment, a different multi-arm system for tracking and compiling multiple, small surgical markers is provided. The system comprises at least three robotic arms based on a single point of origin with a central control unit coordinating the movement of the arms. At least two of the robotic arms carries a surgical tool and one of the robotic arms carries a navigation/tracking camera/sensor. Optionally, a surgical marker may be placed on the arms carrying the surgical tools or on the surgical tools themselves. One or more small surgical markers may be placed in the surgical field on the patient anatomy of interest. In one example, one or more small surgical markers are placed on multiple vertebrae or multiple vertebrae aspects. In various examples, the surgical navigation camera may be held and manipulated closer than conventional surgical navigation cameras, for example at 1 meter or less from the patient anatomy of interest. In various scenarios, the robotic arms holding surgical tools may be deployed into the surgical field to carry out various surgical procedures, including robotic spinal surgery procedures. This system is able to track the patient anatomy of interest, and optionally multiple surgical tools, without unduly interfering with the surgical field or the surgeon's view of the field, due to the use of several small markers.

Keeping in mind that, despite the fact that the embodiments employ surgical navigation cameras held relatively close to the surgical field (1 meter or less from the anatomy of interest), it is still the case that the use of small markers can be constraining (one of skill in the art understands that larger surgical markers facilitate greater tracking accuracy). Thus, in some embodiments, multiple small markers may be mathematically compiled to virtually present one or more larger markers to the navigation camera. In these embodiments, small markers are used and placed on the patient anatomy of interest and, optionally, on surgical tools or on the robotic arms themselves—in this way, the small markers are not interfering with the view of the anatomy of interest. However, the compilation of the small markers provides added accuracy because the navigation camera (held relatively close to the anatomy of interest, at 1 meter or less) “sees” a larger marker due to the mathematical compilation and, thus, greater accuracy is provided. One of skill in the art will understand that “compilation” of multiple small markers refers to a mathematical transformation that, for navigation purposes, turns multiple small markers into one larger marker which can facilitate higher accuracy.

In another embodiment, another form of small marker's tracking can be described. As mentioned, one of the significant drawbacks in the basic technique of markers tracking is the need to view all the relevant markers in one single camera frame. So as an example, in standard surgical technique, the camera needs to see in one view the patient markers, the robotic markers and the tools markers. As said, in regular navigation systems each marker size can be of a minimum of 10 cm and all three mentioned markers together creates now a very large array that the navigation camera needs to see in one image. This is a significant problem that is very cumbersome and does not allow the area of surgical navigation to progress. Moreover it forces the navigation system user to place the navigation camera relatively far (e.g., farther than 1.5 m) which is again cumbersome and also effect accuracy. In the current disclosure, because of the system multi arm morphology and the fact that the robotic arm which holds the navigation camera is calibrated and synchronized with the surgical arms on one single rigid chassis, and the entire robotic system is registered to the patient using (e.g., CT, X-ray etc.), the navigational arm can take several single images with each marker independently and robotically and mathematically compile it to one markers array. Therefore, there is no need to see all the markers in one single image. The robotic navigation camera can take one image of a marker or more than robotically moves to take another image of the next or rest of the markers in the field.

Since the coordinates systems are synchronized the controller can calculate the coordinate system transformation from one image to the other and compile it to one image. Here the multiple small markers are not compiled to one marker, but the over coordinates system is compiled to one coordinate system.

In a first aspect, the present disclosure provides a method for compiling a virtual surgical navigation marker for use with a surgical robot having a robotic surgical coordinate space. The method comprising placing at least two physical surgical markers at spaced-apart locations on an anatomy of a patient. A location of each of the physical surgical markers in the robotic surgical coordinate space is then determined, and the locations of each of said surgical markers in the robotic surgical coordinate space are used to mathematically or otherwise calculate the location, e.g., coordinates, of the single virtual surgical marker is mathematically generated based upon the determined of the physical markers. The single virtual marker will be effectively larger than any one of the physical markers, and tracking of the virtual marker will provide greater accuracy in navigation than tracking any one of the smaller physical markers alone.

In some embodiments, the at least two surgical markers each have a maximum dimension of 5 cm, usually 3 cm, and sometimes 2 cm.

In some embodiments, the at least two surgical markers are spaced apart by a minimum distance of at least 1 cm, usually at least 2 cm, more usually at least 3 cm, and sometimes at least 5 cm, at least 10 cm, or greater.

In some embodiments, placing the at least two surgical markers at spaced-apart locations on the anatomy of the patient comprises implanting the surgical markers in bone.

In some embodiments, determining the location of each of the at least two surgical markers in the surgical coordinate space comprises scanning the anatomy and providing the scanned locations to a controller of the surgical robot.

In some embodiments, the at least two surgical markers are implanted on the patient's anatomy of by robotic arms of the surgical robot and the location of each of the at least two surgical markers in the surgical coordinate space is determined by a controller of the surgical robot based upon kinematically tracked positions of the robot arm at the time each of the at least two surgical markers is implanted.

In some embodiments, the controller deploys at least one sensor to scan the locations of the at least two surgical markers on the anatomy of the patient and determines a location of the single virtual surgical marker based upon said scanned locations.

In some embodiments, the controller positions one or more working robotic arms which carry surgical tools based upon based upon a scanned location of the mathematically generated single virtual surgical marker.

In a second aspect, the present disclosure provides methods for sensor-based tracking of a virtual surgical navigation marker. The virtual surgical navigation marker comprises coordinates in a surgical robotic coordinate space where said coordinates are mathematically generated based upon the determined of at least two spaced-apart physical markers implanted on a patient prior to or during a robotic surgery performed by a surgical robot in the robotic surgical coordinate space. The sensor-based tracking method comprises sensing the locations of each of the at least two spaced-apart physical markers in the robotic surgical coordinate space and calculating a location of the virtual marker in the robotic surgical coordinate space based upon the sensed locations of each of the at least two spaced-apart physical markers in the robotic surgical coordinate space. Sensing and calculating are performed by a controller in communication with the surgical robot.

In some embodiments, the at least two surgical markers each have a maximum dimension of 5 cm, usually 3 cm, and sometimes 2 cm.

In specific instances, the at least two surgical markers are spaced-apart by a minimum distance of at least 2 cm, more usually at least 3 cm, and sometimes at least 5 cm, at least 10 cm, or greater.

In some embodiments, sensing the locations of each of the at least two spaced-apart physical markers in the robotic surgical coordinate space comprises optically tracking the physical markers with a camera which feeds data to the controller.

In some embodiments, the camera is mounted on an arm of the surgical robot.

In some embodiments, the controller repositions the surgical arm carrying the camera to capture all physical markers in the camera's field of view. Such repositioning occurs in response to the controller determining that at least one of the at least two physical markers is not in the camera's field of view.

In some embodiments, the controller positions one robotic arm to locate a surgical tool at an anatomic target location based upon the calculated location of the virtual marker in the robotic surgical coordinate space.

In a third aspect, the present disclosure provides surgical robotic system comprising a surgical robot including at least two robotic arms and a sensor mounted on one of at least two robotic arms. The sensor is configured to track surgical navigation markers on a patient's anatomy, and a controller is operatively coupled to the surgical robot and is configured to (a) receive initial location data for at least two of the surgical navigation markers at the outset of a robotic surgical procedure, (b) mathematically generate a single virtual surgical marker based upon the initial locations of each of the at least two small surgical markers at the outset of the robotic surgical procedure, (c) track positions of the at least two of the surgical navigation markers over time after the surgical procure has started; and (d) calculate the location of the single virtual surgical marker based upon the tracked locations of each of the at least two small surgical markers.

In some embodiments, the initial location data for at least two of the surgical navigation markers is based upon a scan of the patient anatomy.

In some embodiments, the scan of the patient anatomy comprises a computerized tomography (CT) scan performed prior to the procedure.

In some embodiments, the controller is configured to determine initial locations each of the at least two surgical markers in the surgical coordinate space based upon kinematically tracked positions of the robot arm carrying the sensor at the time each of the at least two surgical markers is implanted.

In some embodiments, the controller is configured to deploy at least one sensor to scan the locations of the at least two surgical markers on the anatomy of the patient and to calculate a location of the single virtual surgical marker based upon said scanned locations.

In some embodiments, the surgical robotic system comprising at least three robotic arms wherein at least two of the at least three robotic arms are configured to position a surgical tool and wherein at least one of the at least three robotic arms hold a surgical navigation camera.

### BRIEF DESCRIPTION OF THE DRAWINGS

FIG. 1 shows a multi arm robotic system suitable for use with the navigation markers of the present disclosure.

FIGS. 2A and 2B show different ways that individual physical markers might be compiled into larger virtual surgical markers in accordance with he principles of the present disclosure.

FIGS. 3A and 3B illustrate how a robotically controlled camera may be positioned when tracking two or more physical navigation markers to track a virtual navigation marker in accordance with the principles of the present disclosure.

FIG. 4 is a chart setting forth steps in a first optional method of the present disclosure.

FIG. 5 is a chart setting forth steps in a second optional method of the present disclosure.

FIG. 6 is a chart setting forth steps in a third optional method of the present disclosure.

### DETAILED DESCRIPTION

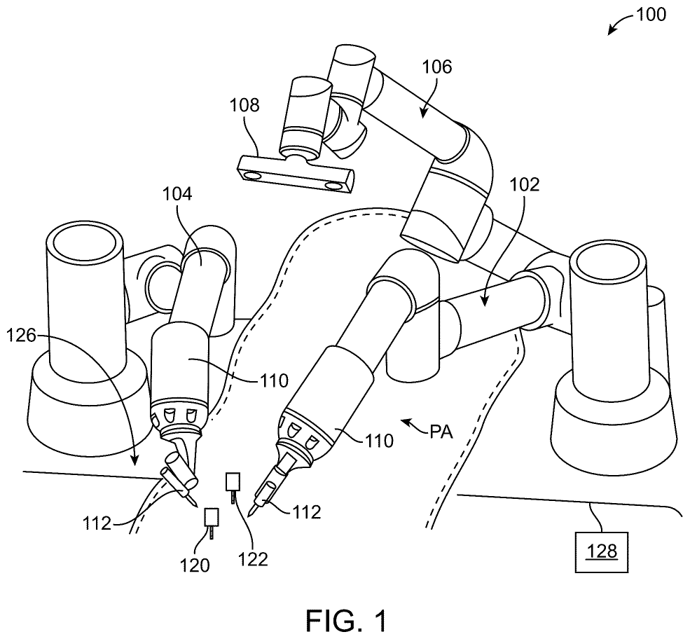

The novel surgical robotic navigation markers and tracking methods of the present disclosure are useful with a variety of surgical robots such as the exemplary multi-arm surgical robotic system 100 illustrated in FIG. 1. The multi-arm surgical robotic system 100 typically comprises at least one “working” arm configured to hold an interventional surgical tool adapted to perform a surgical task, such as implanting screws and other components, boring into bone, suturing tissue, plicating tissue, and the like.

As illustrated in FIG. 1, a surgical robotic system 100 includes two working arms 102 and 104, but suitable robotic systems could include, three, four, or more arms, as shown for example in commonly owned PCT application PCT/IB2023/056911, entitled “Integrated Multi-Arm Mobile Surgical Robotic System,” the full disclosure of which is incorporated herein by reference. The working robotic arms 102 and 104 each have an end effector 110 which carries or incorporates a surgical tool 112.

Suitable robotic systems will typically further comprise at least one surveillance arm 106 used to carry and operate at least one camera or other sensor 108 which is configured to view or sense patient anatomy PA including surgical robotic navigation markers 120 and 122 which been attached to bony or other patient anatomy.

The robotic arms 102, 104, and 106 are typically mounted on an “integrated” mechanical chassis 126 which provides a common surgical space and coordinate system in which a controller 128 can manipulate the arms and associated tool, cameras, and sensors. Usually but not necessarily the controller 128 will be mounted on or otherwise incorporated into the common cart or chassis 126 with the other components of the surgical robotic system 100. The robotic system 100 typically further comprises a display or other interface (not shown) that allows the surgeon to manually instruct or otherwise interact the controller 128 during a surgical procedure. The surgical robotic controller 128 will typically be configured to synchronize and coordinate motion of the robotic arms and related end effectors to perform a desired surgical procedure.

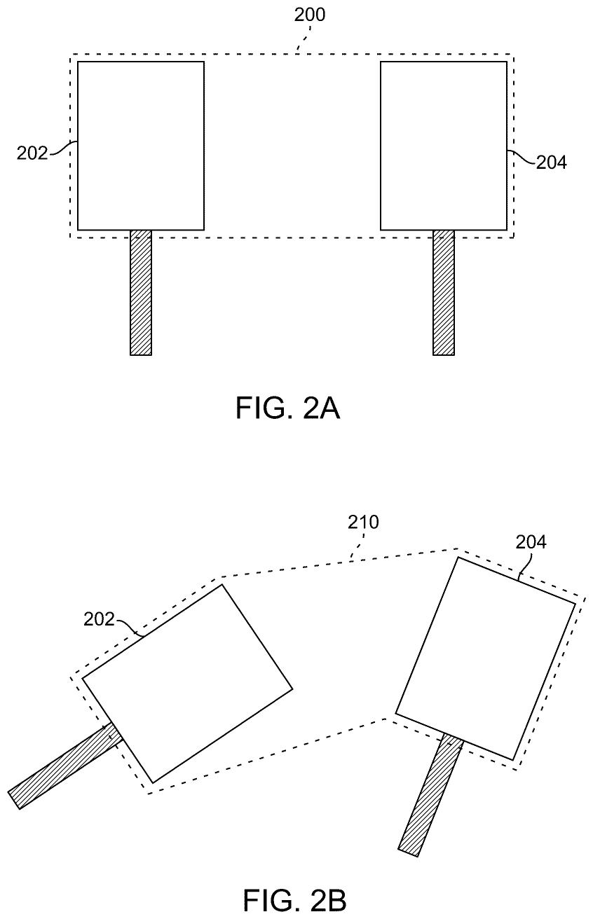

The surgical robotic navigation markers 120 and 122 are typically small, e.g., having a maximum dimension of about 5 cm or less, often about 3 cm or less, sometimes about 2 cm or less. While each marker will typically be configured function as a “stand alone marker,” two or mare markers will be arranged as an “array of markers” which define the dimensions of the virtual markers of the present disclosure. As shown in FIG. 2A, two or more markers 202 and 204 can be implanted at spaced-apart locations on a patient anatomy to define a larger virtual marker 200 having a generally rectangular periphery 200. As shown in FIG. 2B, the same two markers 202 and 204 can be implanted at different spaced-apart locations on the patient anatomy to define a larger virtual marker 210 having a different, irregular periphery 200.

The scanned locations of the physical markers can be used to calculate the parameters of a virtual marker in at least three ways. In a first example, physical marker locations denoted M₁ and M₂ are transformed into a virtual marker M₃ as follows:

The markers M1 and M2 are attached to the spine, patient is undergoing the CT scan.

Let ^{M1}T_{M2} be a transformation from M2 to M1. It can be calculated from the CT scan. Assume M1 marker has points p1, p2 and p3, and M2 has points p4, p5 and p6.

In order to able to track M3 we need to know the location of p1, p2, p3, p4, p5 and p6 in some common coordinate space, e.g., in M1. The p1, p2 and p3 are trivial, as they are already in M1 coordinate space. The p4, p5 and p6 can be calculated in the following way:

$$\begin{matrix}{p}_{4}^{{M}_{1}} {=}^{{M}_{1}} {T}_{{M}_{2}} \times {p}_{4}^{{M}_{2}} \\ {p}_{5}^{{M}_{1}} {=}^{{M}_{1}} {T}_{{M}_{2}} \times {p}_{5}^{{M}_{2}} \\ {p}_{6}^{{M}_{1}} {=}^{{M}_{1}} {T}_{{M}_{2}} \times {p}_{6}^{{M}_{2}}\end{matrix}$$

Thus M3 will be defined by the vector which can be tracked:

$$( \begin{matrix}{p}_{1}^{{M}_{1}} \\ {p}_{2}^{{M}_{1}} \\ {p}_{3}^{{M}_{1}} \\ {p}_{4}^{{M}_{1}} {=}^{{M}_{1}} {T}_{{M}_{2}} \times {p}_{4}^{{M}_{2}} \\ {p}_{5}^{{M}_{1}} {=}^{{M}_{1}} {T}_{{M}_{2}} \times {p}_{5}^{{M}_{2}} \\ {p}_{6}^{{M}_{1}} {=}^{{M}_{1}} {T}_{{M}_{2}} \times {p}_{6}^{{M}_{2}}\end{matrix} )$$

In a second example, the virtual compiled marker now is compiled from two markers attached to the end effectors of the robots. As both robots have a common root, the transformation ^{M1}T_{M2} can be calculated using forward kinematics for both robotics arms, taking into consideration the known transformation between arm, meaning

$${}^{{M}_{1}} {T}_{{M}_{2}} = {DK}_{{Arm}_{1}} \times {}^{{Arm}_{1}} {T}_{{Arm}_{2}} \times {DK}_{{Arm}_{2}}^{{- 1}_{}}$$

Where DK_{Arm1} and DK_{Arm2} are forward kinematics calculation from the base of the robot to the navigation marker. From this point, calculation of the compiled marker is the same as described above.

In a third example, after compiling the two or more small markers into a virtual marker as described in either of the above examples, the tracking camera may reach a position where it is unable to see both smaller markers that must be observed to track the virtual marker. In such cases, the controller will usually be able to reposition the surveillance arm to move the camera to a position where it can view both smaller markers simultaneously. In rare instances, however, there may be no available vantage point where both smaller markers can be observed simultaneously (e.g., the working robotic arms block the necessary lines of sight). In these rare instances, the controller can move the camera to sequentially obtain the necessary location data for each of the two (or more) smaller markers to allow reconstruction and tracking of the virtual marker. The controller can determine each of the two or more sequential camera positions based on the kinematically tracked positions of the surveillance arm which carries the camera. All robotic arms, including the surveillance arm(s) are accurately synchronized with the markers and the patient anatomy (e.g., vertebrae), allowing the controller to accurately position the camera to capture the two (or more) small markers in separate views.

The accurate kinematic further allows the controller to merge the two or more images to form the single virtual marker. Separate tracking of the smaller elements also allows tracking even if the smaller markers do not all face the same direction and/or not visible from the same direction.

Let p1, p2, p3, p4, p5, p6 be 6 single points of the marker M. Let's assume that in position L1, camera can see points p1, p2, p3 and in position L2, camera can see the points p4, p5, p6 of the marker M. In position L1, the camera can see the points p1, p2, p3, so the origin Morig1 of the marker M can be calculated, similar, in position L2, the camera can see the points p4, p5, p6, so the origin Morig2 of the marker can be calculated.

The camera is attached to the robot, so by using robot kinematics we can calculate transformation T: L2→L1.

Now, we can transfer both origins Morig1 and Morig2 into the same coordinate system, for example, the coordinate system of Morig1:

$${M}_{orig 2}^{′} = T \cdot {M}_{orig 2}$$

Now we have redundant information: Morig1 and Morig2 are the same point tracked from a different angle. By averaging these 2 we have a more accurate location of the marker M.

In all cases, the larger virtual marker can be followed in the surgical coordinate space by using the camera 108 or other sensor to simultaneously track both of the physical markers 120 and 122 that were used to define the virtual marker. Such tracking assumes that the physical markers will not move relative to each during a procedure. Should either or both of the physical markers be dislodged or displaced, their usefulness for tracking the virtual marker will end.

In a first representative embodiment, the robotic arms 102 and 104 implant the single physical markers 120 and 122 at specific location in space that create a precise and predetermined pattern of a large virtual or compiled marker. Usually but not necessarily, each physical markers 120 and 122 will have a size less than that needed to achieve a desired accuracy if used as a “stand alone marker,” typically having a maximum dimension below about 5 cm. Accordingly, when the robotic arm with camera is tracking one marker only at a common tracking distance, the tracking accuracy is less than desired.

By defining a larger virtual marker from two or more smaller physical markers, tracking accuracy can be improved. For example, two or more single physical markers sized smaller than about 5 cm can be compiled into a virtual marker with an effective size greater than 10 cm, significantly improving tracking accuracy.

Accurate tracking of multiple physical markers that comprise the larger virtual marker relies on accurate kinematic tracking of the cameras and other sensors that follow the physical markers as well as the working surgical arms 102 and 104 if used to implant the physical markers. By mounting all the robotic arms on a single same rigid chassis or multiple rigidly attached chassis components, robotic calibration and synchronization of all robot arms 102, 104, and 106 can be significantly improved. In addition, another method can be used where the controller can send the robotic arms to a non-random, predetermined deterministic positions in space in it the robots were calibrated in the factory and by that to use the “repeatable accuracy” of the robotic arms. One familiar in the art knows that a ‘repeatable accuracy’ of industrial robots is much higher than in any random location in space what will insure very high accuracy of the positions of the arms.

One of skill in the art will understand that this technique is significantly enhanced with a highly accurate multi-arm system in which the robotic arms are robotically coordinated from a single rigid chassis. In a multi-arm system in which, for example, each robotic arm is placed on a separate cart, the synchronization between the arms must be done by another navigation/tracking system which of course now has its own intrinsic inaccuracy making such systems less preferred for use in the present technology.

In a second embodiment of the present technology where a designated anatomical field is relatively large (e.g., human spine), one small marker (e.g., smaller than 5 cm) might be insufficient to achieve the level of accuracy for spine surgery. But as explained above, in spine surgery it is very limiting to place large markers on the delicate and flexible vertebrae. In the said example, one possible option is to place two more small markers, for example one marker on one vertebra and a second small marker several vertebrae above or below.

As described above each marker can be tracked by the camera as a ‘stand-alone’ marker with its relative accuracy coming from its relatively small size. But in the suggested system, the camera is robotically maneuvered and controlled and registered to the anatomy together with the rest of the robotic arms (e.g., through CT, X-ray scan). Accordingly, the system controller knows the camera's location in space and the markers' location in space. Also at the beginning the controller will capture and save first preliminary position of the markers as basic reference.

The controller can position the robotic arm with the camera in a precise and specific location in space in relation to the two markers. In this specific location in space the two small markers create a specific pattern of the large, compiled marker with a high accuracy. The controller knows and governs this and can captures this large marker.

A third representative embodiment is a possible combination of the two example above. Meaning, in the surgical field where there are one or more small markers fixed to the anatomy and registered to it, thus creating together a large, compiled marker, one or more additional smaller markers are introduced by one or more surgical robotic arms which are also registered to the anatomy. This compiled large marker can be even much larger and can be comprised of two, three, four or more markers covering a very large area and integrating together into a very large marker (larger than 20 cm).

A fourth representative embodiment is not a compilation of a large marker from two or more small markers but rather a compilation of several coordinates systems of several small markers. Since the coordinate system of the robotic arm which holds and maneuvers the navigation camera is coordinated and synchronized with to the rest of the robotic arms as well as being registered to the anatomy and the small patient markers are also registered to the patient anatomy, the single controller knows at any given point the location of all the robotic arms and markers.

Knowing this, in this system the navigation camera doesn't have to see all the markers together in a single image. The robotic arm can position the camera in an optimal position to capture one or more small markers and then move it to capture the rest. This way the robotic system can capture several small markers and compile all their coordinate systems.

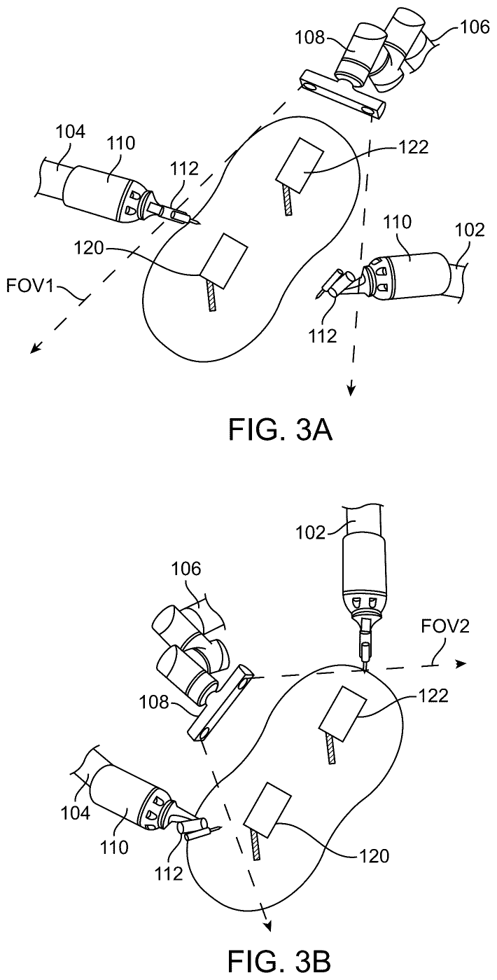

Referring now to FIGS. 3A and 3B, tracking of physical markers 120 and 122 requires that the camera 108 have a field of view FOV1 which is large enough and oriented properly so that the camera can simultaneously view both physical markers. As shown in FIG. 3A, the camera 108 is aligned one longitudinally with the two markers 120 and 122, and the robot arms 102 and 104 are to the sides so that the field of view is uninterrupted. In contrast, in FIG. 3B the arms 102 and 104 have moved so that they would obscure the view of the camera 108 if positioned as shown in FIG. 3A, Thus, in order to have a field of view FOV2 which is sufficiently large and oriented to simultaneously view both physical markers 120 and 122, the surveillance arm 106 needs to reposition the camera 108 to view across the two markers. For proper performance of the present technology, it is necessary that both markers 120 and 122 be simultaneously viewed by the camera 108 to allow computation of the virtual marker as described elsewhere herein.

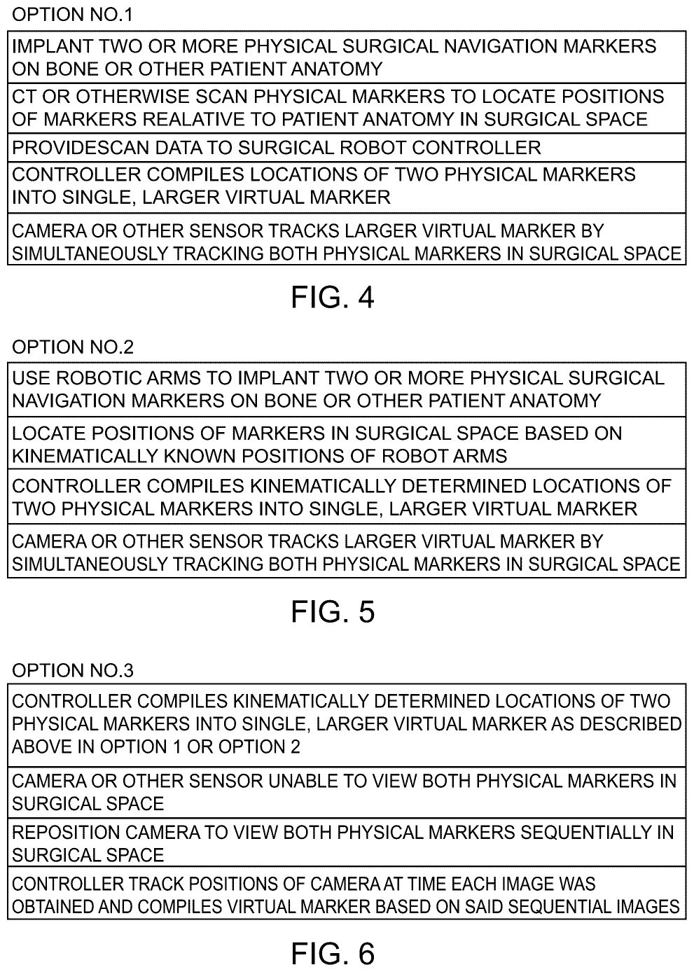

Referring now to FIG. 4, the steps in a first optional method for creating and tracking virtual markers in accordance with the principles of the present technology will be described. Two or more physical surgical navigation markers our first implanted on bone or other patient anatomy. This could be done using the surgical robot but will more likely be done in a pre-robotic surgical procedure, allowing the physician to scan the markers and register them within a surgical coordinate space. Scanning will usually use a CT scanner, but could alternatively use fluoroscopic, ultrasound, or other imaging modalities. After the physical markers are scanned, the scanning data is transferred to a controller of the surgical robot, and the controller of calculates or compiles the single, larger virtual marker based upon the locations of the two physical markers. The camera or other sensor of the surgical robot can then track the larger virtual markers by simultaneously tracking both physical markers in the surgical space as the robotic surgery proceeds.

Referring now to FIG. 5, The steps in a second optional method for creating and tracking virtual markers in accordance with the principles of the present technology will be described. The working robotic arms of the surgical robot are used to implant two or more physical surgical. navigation markers. The patient's anatomy will have been previously registered with the robotic coordinate system so the locations of the implanted markers can be kinematically determined by the surgical robot controller. The controller then compiles the positions of the two physical markers into a single virtual marker, and the robotic camera or other sensor can then track the virtual marker by simultaneously tracking the two physical markers to determine movements in the patient anatomy.

Referring now to FIG. 6, The steps in a third optional method for creating and tracking virtual markers in accordance with the principles of the present technology will be described. The controller of the surgical robot compiles the kinematically determined locations of the two physical markers into a single, larger virtual marker as described with reference to FIG. 5 or 6 above. If the camera or other sensor is unable to view both physical markers and the surgical space simultaneously, the camera will be repositioned to view both physical markers sequentially. The controller tracks the position of the virtual marker based on the sequentially obtained images of the two physical markers.

Although certain embodiments or examples of the disclosure have been described in detail, variations and modifications will be apparent to those skilled in the art, including embodiments or examples that may not provide all the features and benefits described herein. It will be understood by those skilled in the art that the present disclosure extends beyond the specifically disclosed embodiments or examples to other alternative or additional examples or embodiments and/or uses and obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments and examples may be made and still fall within the scope of the present disclosure. Accordingly, it should be understood that various features and aspects of the disclosed embodiments can be combined with or substituted for one another in order to form varying modes or examples of the present disclosure. Thus, it is intended that the scope of the present disclosure herein disclosed should not be limited by the particular disclosed embodiments or examples described above. For all of the embodiments and examples described above, the steps of any methods for example need not be performed sequentially.

What is claimed is:

1. A system for robotically, navigationally and mathematically compiling multiple small surgical markers comprising:
a robotic surgery system comprising at least two robotic arms mounted on a single chassis containing a central controller, wherein at least one of the at least two robotic arms positions and/or holds a surgical tool and at least one of the at least two robotic arms holds a surgical navigation camera or sensor; and
at least two small surgical markers placed on anatomy of interest of a patient;
wherein the at least two small surgical markers are registered to the anatomy and navigationally and mathematically compiled by a processor of the central controller such that the compiled larger marker is used by the navigation system.

2. The system of claim 1, further comprising one or more further small surgical markers placed on each of the at least one robotic arms which hold a surgical tool.

3. The system of claim 1, comprising at least three robotic arms wherein at least two of the at least three robotic arms each position and/or hold a surgical tool and wherein at least one of the at least three robotic arms holds a surgical navigation camera.

4. The system of claim 1, wherein the small surgical markers are each less than 5 cm in size.

5. The system of claim 1, wherein the surgical navigation camera is positioned by the robotic surgery system at a distance of less than one meter from the patient anatomy of interest.

6. The system of claim 1, wherein the small surgical markers are compiled mathematically into one larger surgical marker by virtue of the central controller knowing the position of the robotic arms and the small surgical markers in relation to the anatomy.

7. The system of claim 1, wherein the navigation camera or sensor employs technology selected from the group consisting of CT, MRI, magnetic and laser sensors.

8. The system of claim 1, wherein the at least two small surgical markers are active markers.

9. The system of claim 1, wherein the at least two small surgical markers are passive markers.

10. The system of claim 1, wherein the robotic surgery system is mobile.

11. A method for navigationally and mathematically compiling multiple surgical markers comprising:
providing a robotic surgery system comprising at least two robotic arms mounted on a single chassis containing a central controller, wherein at least one of the at least two robotic arms positions and/or holds a surgical tool and at least one of the at least two robotic arms holds a surgical navigation camera or sensor;
placing at least two small surgical markers on anatomy of interest of a patient; and
mathematically compiling the at least two small surgical markers such that a larger surgical marker is presented to the navigation camera or sensor.

12. The method of claim 11, wherein the at least two small surgical markers are less than 5 cm in size.

13. The method of claim 11, wherein the navigation camera is held at a distance of less than one meter from the patient anatomy of interest.

14. A method for compiling a virtual surgical navigation marker for use with a surgical robot having a robotic surgical coordinate space, said method comprising:
placing at least two surgical markers at spaced-apart locations on anatomy of a patient;
determining a location of each of the at least two surgical markers in the robotic surgical coordinate space; and
mathematically generating a single virtual surgical marker based upon the determined locations of each of the at least two surgical markers.

15. The method of claim 14, wherein the at least two surgical markers each have a maximum dimension of 5 cm.

16. The method of claim 14, wherein the at least two surgical markers are spaced-apart by a minimum distance of 1 cm.

17. The method of claim 14, wherein placing the at least two surgical markers at spaced-apart locations on the anatomy of the patient comprises implanting the surgical markers in bone.

18. The method of claim 14, wherein determining the location of each of the at least two surgical markers in the surgical coordinate space comprises scanning the anatomy and providing the scanned locations to a controller of the surgical robot.

19. The method of claim 14, wherein the at least two surgical markers are implanted on the patient's anatomy of by robotic arms of the surgical robot and the location of each of the at least two surgical markers in the surgical coordinate space is determined by a controller of the surgical robot based upon kinematically tracked positions of the robot arm at the time each of the at least two surgical markers is implanted.

20. The method of claim 14, wherein the controller deploys at least one sensor to scan the locations of the at least two surgical markers on the anatomy of the patient and determines a location of the single virtual surgical marker based upon said scanned locations.

21. The method of claim 14, wherein the controller positions one or more working robotic arms which carry surgical tools based upon based upon a scanned location of the mathematically generated single virtual surgical marker

22. A method for sensor-based tracking of a virtual surgical navigation marker comprising at least two spaced-apart physical markers during a robotic surgery performed by a surgical robot in a robotic surgical coordinate space, said method comprising:
sensing the locations of each of the at least two spaced-apart physical markers in the robotic surgical coordinate space;
calculating the location of the virtual marker in the robotic surgical coordinate space in a controller of the surgical robot based upon the sensed locations of each of the at least two spaced-apart physical markers in the robotic surgical coordinate space.

23. The method of claim 22, wherein the at least two surgical markers each have a maximum dimension of 5 cm.

24. The method of claim 22, wherein the at least two surgical markers are spaced-apart by a minimum distance of 1 cm.

25. The method of claim 22, wherein sensing the locations of each of the at least two spaced-apart physical markers in the robotic surgical coordinate space comprises optically tracking the physical markers with a camera which feeds data to the controller.

26. The method of claim 25, wherein the camera is mounted on an arm of the surgical robot.

27. The method of claim 25, wherein if the controller determines that at least one of the at least two physical markers is not in the camera's field of view, the controller repositions the surgical arm carrying the camera to capture all physical markers in the camera's field of view.

28. The method of claim 22, wherein the controller positions one robotic arm to locate a surgical tool at an anatomic target location based upon the calculated location of the virtual marker in the robotic surgical coordinate space

29. A surgical robotic system comprising:
a surgical robot including at least two robotic arms; and
a sensor mounted on one of at least two robotic arms, said sensor configured to track surgical navigation markers on a patient's anatomy;
a controller configured to (a) receive initial location data for at least two of the surgical navigation markers at the outset of a robotic surgical procedure, (b) mathematically generate a single virtual surgical marker based upon the initial locations of each of the at least two small surgical markers at the outset of the robotic surgical procedure, (c) track positions of the at least two of the surgical navigation markers over time after the surgical procure has started; and (d) calculate the location of the single virtual surgical marker based upon the tracked locations of each of the at least two small surgical markers.

30. The system of claim 29, wherein the initial location data for at least two of the surgical navigation markers is based upon a scan of the patient anatomy.

31. The system of claim 30, wherein the scan of the patient anatomy comprises a computerized tomography (CT) scan performed prior to the procedure.

32. The system of claim 29, wherein the controller is configured to determine initial locations each of the at least two surgical markers in the surgical coordinate space based upon kinematically tracked positions of the robot arm carrying the sensor at the time each of the at least two surgical markers is implanted.

33. The system of claim 29, wherein the controller is configured to deploy at least one sensor to scan the locations of the least two surgical markers on the anatomy of the patient and to calculate a location of the single virtual surgical marker based upon said scanned locations.

34. The system of claim 29, comprising at least three robotic arms wherein at least two of the robotic arms are configured to position a surgical tool, and wherein at least one of the at least three robotic arms holds a surgical navigation camera.
